(19) United States
(12) Patent Application Publication
(10) Pub. No.: US 2011/0160450 A1
(43) Pub. Date: Jun. 30, 2011

(54) EXPLOSIVE

(75) Inventor: Ulrich Baudis, Alzenau (DE)

(73) Assignee: DURFERRIT GMBH, Mannheim (DE)

(21) Appl. No.: 13/060,617

(22) PCT Filed: Jul. 24, 2009
(86) PCT No.: PCT/EP2009/005377

§ 371 Date: Feb. 25, 2011

(30) Foreign Application Priority Data
Aug. 30, 2008 (DE) 10 2008 045 192.4

(51) Int. Cl. C07D 487/22; C07D 487/12

(52) U.S. Cl. 540/471; 544/194

(57) ABSTRACT

The invention relates to an explosive comprising 2,6,10-trinitro-s-heptazine and/or derivatives thereof as an active chemical compound.

### CROSS REFERENCE TO RELATED APPLICATION

The present application is a U.S. National Stage of International Application No. PCT/EP2009/005377, filed Jul. 24, 2009, designating the United States and claiming priority to German Application No. 10 2008 045 192.4, filed Aug. 30, 2008.

### FIELD OF THE INVENTION

The invention relates to an explosive.

For the production of known modern explosives, for example trinitrotoluene (TNT) or hexogen (HMX), a nitrating reaction is used to insert nitro groups, meaning NO₂ groups, into a carbon-containing and nitrogen-containing organic molecule. In this way, the oxygen required for the combustion, respectively for the explosive-type conversion of the explosive, is anchored in the active chemical compound of the explosive material itself. The so-called explosives are high-energetic materials which are used together with other explosive materials, for example octogen (RDX), in numerous industrial applications.

One disadvantage of these explosives is that the explosive materials themselves are very poisonous, wherein TNT, for example, can cause allergic reactions when coming in contact with skin and hexogen even has a carcinogenic effect.

A further disadvantage is that explosives of this type, for example TNT, do not have a balanced oxygen-carbon balance and that the starting materials for producing the explosive are dangerous.

### SUMMARY OF THE INVENTION

It is the object of the present invention to provide a high-energetic, easy to handle explosive which can be used in numerous industrial applications.

The explosive according to the invention contains 2,6,10-trinitro-s-heptazine and/or its derivatives as active chemical compound.

The active chemical compound in general can form an explosive or can form a component of the explosive.

In addition to or as an alternative to 2,6,10-trinitro-s-heptazine, the explosive according to the invention can furthermore comprise as chemically active compound 2-amino-6,10-dinitro-s-heptazine or 2,6-diamino-10-nitro-s-heptazine, meaning derivatives of the 2,6,10-trinitro-s-heptazine.

One essential advantage of using 2,6,10-trinitro-s-heptazine as chemically active compound in the explosive according to the invention is that this compound has a completely balanced oxygen-carbon balance. When initiating a decomposition of the explosive, the 2,6,10-trinitro-s-heptazine, having the chemical formula C₆N₁₀O₆, quantitatively decomposes into carbon monoxide (CO) and nitrogen according to the following equation

C₆N₁₀O₆→6 CO+5 N₂

This total decomposition into thermodynamically stable gases means that 2,6,10-trinitro-s-heptazine is a chemically active compound with high energy potential. Thus, 2,6,10-trinitro-s-heptazine, in the same way as its derivatives, represents a high-explosive material which can be used for a broad spectrum of applications, in particular also as a propellant charge.

The explosive according to the invention, which is composed of the aforementioned chemically active compounds, can furthermore be used quite advantageously as a gas generator or a gas producer. Gas producers of this type are utilized, for example, in the manufacture of vehicles, such as for the production of airbags, belt-tightening systems, and the like.

These applications for the explosive according to the invention in particular are possible because the chemically active compounds are not poisonous, are not dangerous, and are easy to handle.

An additional and essential advantage of the invention is that chemically inert and toxicologically harmless materials can be used for producing 2,6,10-trinitro-s-heptazine and/or its derivatives 2-amino-6,10-dinitro-s-heptazine and 2,6-diamino-10-nitro-s-heptazine.

Triamino-s-heptazine, also known under the name of melem, can be used as starting material for this chemically active compound. Other designations for melem are triamino-tri-s-triazine, cyamelluric triamides or 1,3,4,6,7,9,9b-heptaazaphenalene-2,5,8-triamines.

The polymer of 2,6,10-triamino-s-heptazine, which is also known under the name melon, is a different starting material that can be used for the chemically active compound of the explosive according to the invention.

It has proven to be especially advantageous to use as the starting material for the chemically active compound according to the invention the regenerating means for nitrocarburizing salt baths that is produced by the company HEF-Durferrit GmbH, Mannheim (Germany) and is marketed under the registered trademark REG1®, which contain a mixture of melon and melem.

One essential advantage of melem, melon, as well as REG1® is that these substances are chemically inert and non-poisonous.

For producing 2,6,10-trinitro-s-hepatzin or its derivatives, the substances melem, melon or REG1® are generally nitrated by using suitable nitrating reagents. If REG1® is used as starting material, the nitration process results in a mixture of 2,6,10-trinitro-s-heptazine, its derivatives and a different chemically active compound that is generally obtained with the aid of a reaction of REG1® with nitrating acid or nitrating reagents and is henceforth referred to as nitro-melon.

This nitro-melon as a new type of chemically active compound can be used by itself as the chemically active compound for the explosive according to the invention or in a mixture with 2,6,10-trinitro-s-heptazine and its derivatives.

Nitro-melon for the most part exhibits properties which correspond to those of 2,6,10-trinitro-s-heptazine and its derivatives.

### BRIEF DESCRIPTION OF THE DRAWINGS

In the following, the invention is explained with the aid of examples and representations, which show in:

FIG. 1: The structural formula for trinitro-s-heptazine;

FIG. 2: The structural formula for triamino-s-heptazine;

FIG. 3: The structural formula for tripotassium cyamelurate;

FIG. 4: The structural formula for cyamelluric acid;

FIG. 5: The structural formula for trichlor-s-heptazine (trichlor cyamelluric acid);

FIG. 6: The nitrating of trichlor-s-heptazine;

FIG. 7: The structural formula for melon

FIG. 8: The structural formula for nitro-melon.

### DETAILED DESCRIPTION OF THE EMBODIMENTS

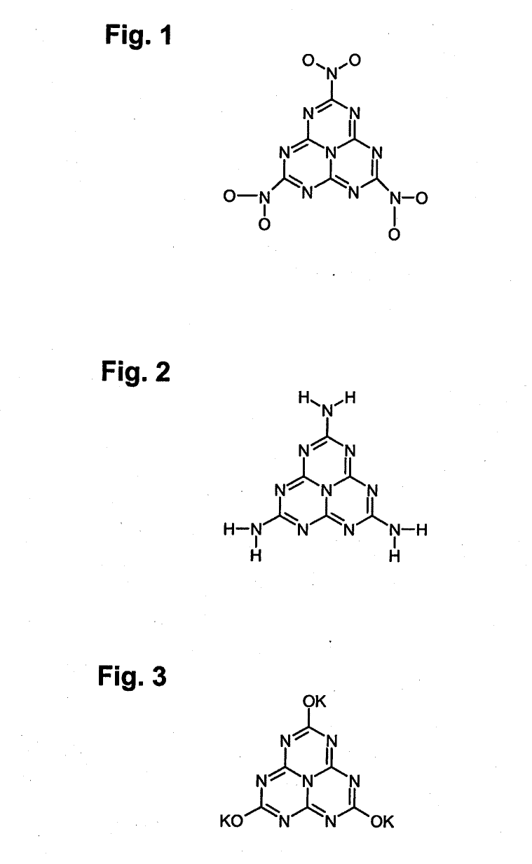

FIG. 1 shows the structural formula for 2,6,10-trinitro-s-heptazine which is used as the chemically active compound in the explosive according to the invention. As an alternative or in addition thereto, derivatives of 2,6,10-trinitro-s-heptazine, namely 2-amino-6,10-dinitro-s-heptazine and 2,6-diamino-10-nitro-s-heptazine, can also be used as chemically active compound for the explosive according to the invention.

This chemically active compound forms an explosive with high energy potential and balanced oxygen-carbon balance, wherein this compound is converted completely to carbon monoxide and nitrogen during an explosion of the explosive material. As a result, the chemically active compound forms propellants and in particular gas producers which can be used, for example, for airbags.

The 2,6,10-trinitro-s-heptazine can be produced through the process of nitrating 2,6,10-trinitro-s-heptazine, also known as melem or triamino-tri-s-triazine.

FIG. 2 shows the structural formula for melem. The nitrating of melem takes place with the aid of known nitrating reactions, for example as described in the publication Organikum, Wiley VCH, Weinheim 2001 (ISBN 3-527-29985-8), pages 188 and following, as well as 358-361. As substances which provide the nitryl cation NO₂⁺, this publication in particular mentions nitric acid or nitrating acid (a mixture of nitric acid and sulphuric acid). The substances 2-amino-6, 10-dinitro-s-heptazine and 2,6-diamino-10-nitro-s-heptazine can be produced in a similar manner with the aid of an incomplete, respectively interrupted, nitrating process. A different synthesis option is the diazotizing and subsequent nitrating of 2,6,10-triamino-s-heptazine.

In place of melem, which is hard to obtain as a pure substance (Juergens, Irran et al., J. Am. Chem. Soc. 2003 125 (34) 10288-10300), an organic regenerating agent for nitrocarburating salt baths called REG1®, which is manufactured by the company HEF-Durferrit GmbH/Mannheim (Germany), can also be used as the starting material for the aforementioned chemically active compounds. REG1® consists of a mixture of melon, a polymer of the triamino-s-heptazine with the gross formula [C₆H₃N₉]ₓ and melem.

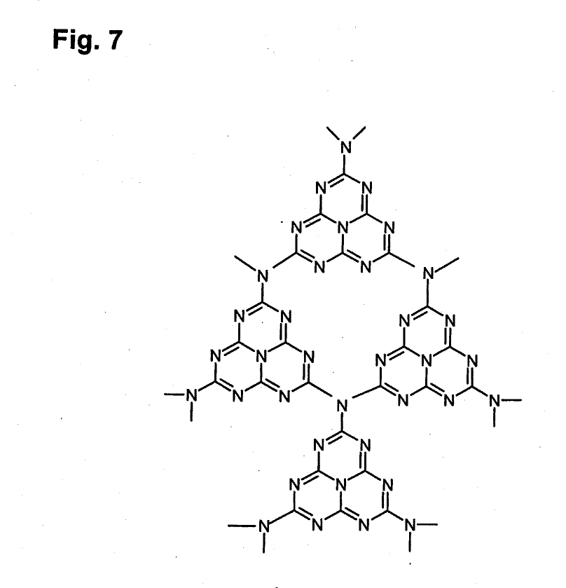

FIG. 7 shows the structural formula for melon. The chemically active compounds are produced through the process of nitrating of REG1®, wherein the aforementioned nitrating reactions can be used in this case as well.

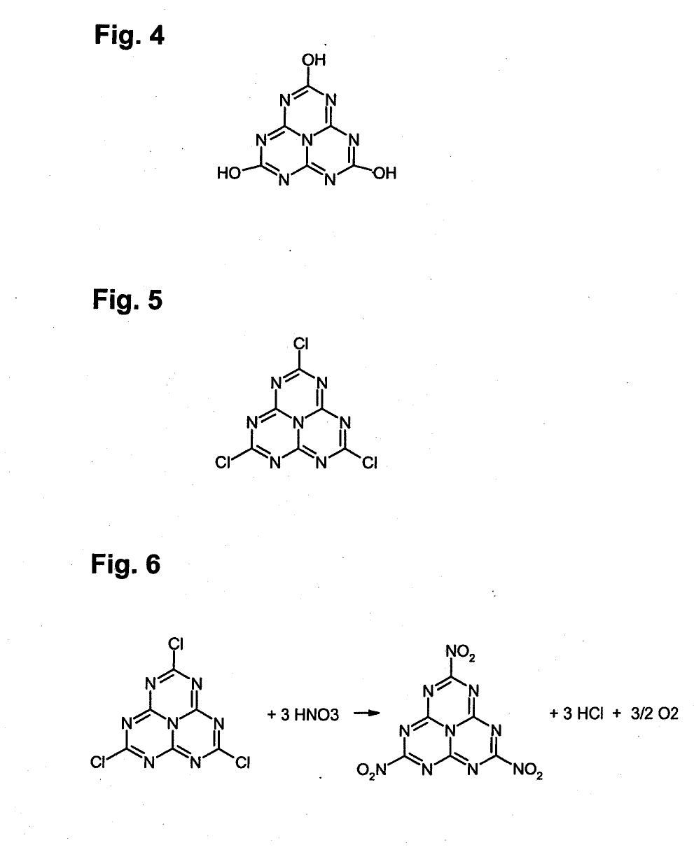

Tripotassium cyamelurate (FIG. 3), the tri-potassium salt of cyamelluric acid or other alkaline salts of the cyamelluric acid (FIG. 4) such as sodium salt or lithium salt, can also be used as alternative starting material for producing the 2,6,10-trinitro-s-heptazine according to the invention. Tripotassium cyamelurate has the gross formula K₃C₆N₇O₃. An extremely good yield of tripotassium cyamelurate is obtained by cooking REG1® for 24 hours or by placing melon in a concentrated potassium hydroxide solution. In the crystallized, non-dried form it contains water of crystallization which can be removed through heating in the vacuum. During the reaction of REG1® with alkali hydroxide, the C-NH-C, respectively the C—N—C, bridges of the component melon, contained in the REG1®, are split while the energy-rich s-heptazine system “C6N7” (alternative designation: tri-s-triazine system or cyamelluric system) is retained. A relatively large amount of the energy-rich tri-s-triazine system in the form of the alkaline salt of the cyamelluric acid can be obtained in this way from melon. The production of K₃C₆N₇O₃ is described in Kroke et al., New. J. Chem., 2002 (26) 508-512. The 2,6,10-trinitro-s-heptazine can then be produced from K₃C₆N₇O₃ with the aid of the following nitration reaction:

C₆N₇(OK)₃+6 HNO₃→C₆N₇(NO₂)₃+3 KNO₃+3 H2O+3/2 O2

The 2,6,10-trinitro-s-heptazine according to the invention can also be obtained through synthesis from the chloride of the cyamelluric acid by substituting nitro groups for the chlorine atoms (FIGS. 5 and 6). Cyamelluric acid chloride (trichlor-tri-s-triazine) is obtained through reaction of alkaline salts of the cyamelluric acid with PC15.The production of cyamelluric acid chloride (C₆N₇Cl₃) is described in Kroke et al., New. J. Chem. 2002 (26) 508-512, as well as in Schroeder and Kober, J. Org. Chem. 1962 (27) 4262-4266.

Other potential starting materials for producing trinitro-s-trazine through the process of nitration can be found in Horvath-Bordon, Kroke et al., Dalton Trans., 2004, 3900-3908.

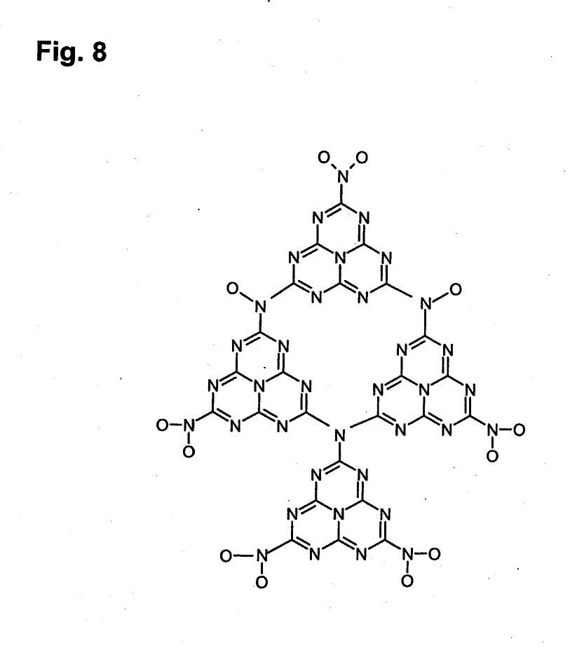

FIG. 8 shows the structural formula of a different chemically active compound, henceforth referred to as nitro-melon, which can be used for the explosive according to the invention as alternative or in addition to the previously mentioned chemically active compounds 2,6,10-trinitro-s-heptazine, 2-amino-6,10-dinitro-s-heptazine and 2,6-diamino-10-nitro-s-heptazine.

The name nitro-melon is selected in reference to the designation for nitro cellulose.

Nitro-melon can be obtained with the aid of known nitration reactions, by allowing nitrating acid or nitrating reagents, for example acetyl nitrate or—which is operationally safer—a mixture of acetic acid anhydride in an environment of water-free acetic acid and with the admixture of concentrated nitric acid to act upon REG1® with good cooling.

Depending on the specific process sequence for the nitrating of REG1®, a mixture of nitro-melon and 2,6,10-trinitro-s-heptazine and its derivatives is obtained, wherein this mixture can be used for the explosive.

1.-7. (canceled)

8. An explosive, comprising:
a chemically active compound including melon treated with nitrating acid or nitrating reagents, melem treated with nitrating acid or nitrating reagents, REG1® treated with nitrating acid or nitrating reagents, 2,6,10-trinitro-s-heptazin and/or a derivative of 2,6,10-trinitro-s-heptazin .

9. The explosive according to claim 8, wherein the chemically active compound comprises 2-amino-6,10-dinitro-s-heptazin as a derivative of 2,6,10-trinitro-s-heptazin.

10. The explosive according to claim 8, wherein the chemically active compound comprises 2,6-diamino-10-nitro-s-heptazin as a derivative of 2,6,10-trinitro-s-heptazin.

11. The explosive according to claim 8, wherein the chemically active compound comprises 2-amino-6,10-dinitro-s-heptazin, and/or a melon treated with nitrating acid or nitrating reagents .

12. The explosive according to claim 8, wherein the chemically active compound comprises REG1® treated with nitrating acid or nitrating reagents.

13. A method of forming a propellant, comprising utilizing the explosive according to claim 8.

14. A method of forming a gas generator, comprising utilizing the explosive according to claim 8.
